(19) United States
(12) Patent Application Publication
(10) Pub. No.: US 2023/0410936 A1
(43) Pub. Date: Dec. 21, 2023

(54) NETWORK APPROACH TO NAVIGATING THE HUMAN GENOME

(71) Applicant: THE REGENTS OF THE UNIVERSITY OF MICHIGAN, Ann Arbor, MI (US)

(72) Inventor: Indika RAJAPAKSE, Ann Arbor, MI (US)

(73) Assignee: THE REGENTS OF THE UNIVERSITY OF MICHIGAN, Ann Arbor, MI (US)

(21) Appl. No.: 18/035,067

(22) PCT Filed: Nov. 3, 2021
(86) PCT No.: PCT/US2021/072199

§ 371 Date: May 2, 2023

Related U.S. Application Data
(60) Provisional application No. 63/109,147, filed on Nov. 3, 2020.

(51) Int. Cl. G16B 5/00; G16B 25/10; G16B 40/20; G16B 45/00

(52) U.S. Cl. CPC G16B 5/00; G16B 25/10; G16B 40/20; G16B 45/00

(57) ABSTRACT

A computer-implemented method is presented for modeling the genome of a cell. The method includes: constructing a graph for a genome, where each node in the graph represents a gene in the genome and each edge in the graph quantifies the relationship between two genes; receiving a first biological sample of a first cell of a subject, where the first cell has a first cell type; determining gene expression data for the first cell from the first biological sample; extracting a first subgraph from the graph using the gene expression data for the first cell, where the subgraph represents the first cell type; receiving a second biological sample of a second cell of a subject, where the second cell has a second cell type; determining gene expression data for the second cell from the second biological sample; extracting a second subgraph from the graph using the gene expression data for the second cell, where the second subgraph represents a second cell type; and comparing the first subgraph to the second subgraph.

### CROSS-REFERENCE TO RELATED APPLICATION

This application claims the benefit of U.S. Provisional Application No. 63/109,147, filed on Nov. 3, 2020. The disclosure of the above application is incorporated herein by reference in its entirety.

### FIELD

The present disclosure relates to techniques for analyzing and manipulating the human genome based on network theory.

### BACKGROUND

A network is a collection of points (nodes or vertices) joined together by lines (edges). A network is commonly referred to as a graph in the mathematics literature. The study of networks is relatively new, but the applications include the internet, social networks and biology, yielding a great deal of useful information. Biological networks can be considered abstract representations of biological systems that capture their essential characteristics. The evolving nature of a network is determined by both the dynamical rules governing the nodes and the flow occurring along each edge.

This disclosure presents a “wiring diagram” or network for the human genome and then derives cell type specific wiring diagrams. This construction enables one to query the genome and explore how perturbations affect the information flow, or navigability, inside the wiring diagram. Furthermore, these networks can help to identify the importance of a gene in a given setting.

This section provides background information related to the present disclosure which is not necessarily prior art.

### SUMMARY

This section provides a general summary of the disclosure, and is not a comprehensive disclosure of its full scope or all of its features.

In one aspect, a computer-implemented method is presented for modeling the genome of a cell. The method includes: constructing a graph for a genome, where each node in the graph represents a gene in the genome and each edge in the graph quantifies the relationship between two genes; receiving a first biological sample of a first cell of a subject, where the first cell has a first cell type; determining gene expression data for the first cell from the first biological sample; extracting a first subgraph from the graph using the gene expression data for the first cell, where the subgraph represents the first cell type; receiving a second biological sample of a second cell of a subject, where the second cell has a second cell type; determining gene expression data for the second cell from the second biological sample; extracting a second subgraph from the graph using the gene expression data for the second cell, where the second subgraph represents a second cell type; and comparing the first subgraph to the second subgraph.

In one embodiment, the importance of nodes in the first and the second subgraphs are quantified using centrality before the step of comparing the first subgraph to the second subgraph.

In another embodiment, the importance of nodes in the first and the second subgraphs are quantified by applying a page rank method to the first and second subgraphs and computing a distance between eigenvectors associated with the first and second subgraphs.

In another aspect, a method is presented for reprogramming cells of a subject. The method includes: receiving a biological sample of a sample cell from the subject, where the sample cell has a given cell type; determining gene expression data for the sample cell from the biological sample; constructing a graph for a genome, where each node in the graph represents a gene in the genome and each edge in the graph quantifies the relationship between two genes; forming an adjacency matrix from the graph; receiving gene expression data for a target cell having a target cell type, where the target cell type differs from the given cell type; computing a regulatory set for a set of transcription factors, where the regulatory set quantifies influence of the transcription factors in the set of transcription factors on a genome; expressing reprogramming of the sample cell to the target cell with a state-space representation of a linear system, where the gene expression data for the target cell serves as an output vector in the state-space representation, the adjacency matrix serves as a state transition matrix, the gene expression data for the sample cell serves as a state vector in the state-space representation, the regulatory set for the given transcription factor serves as an input matrix in the state-space representation, and an input vector in the state-space representation represents the given transcription factor; solving for the input vector in the state-space representation; and manipulating at least one transcription factor in a particular cell of the subject, where the particular cell has the given cell type and the at least one transcription factor is in the input vector.

The graph for a genome may be constructed by representing protein-protein interactions, transcription-DNA interactions and transcription factor-transcription factor interactions with a series of matrices.

In some embodiments, the subset of vertices in the graph is identified using one of degree centrality, closeness centrality, betweenness centrality and eigenvector centrality.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram illustrating a simplified network for the human genome.

FIG. 2 is a diagram of an example embodiment of a network representing the human genome.

FIG. 3 is a flowchart depicting a method for reprogramming cells of a subject using a control system approach.

FIG. 4 is a diagram showing an iterative feedback approach to cell reprogramming.

FIG. 5 is a diagram illustrating cell reprogramming trajectory.

FIG. 6 is a flowchart depicting a method for analyzing the human genome.

Corresponding reference numerals indicate corresponding parts throughout the several views of the drawings.

### DETAILED DESCRIPTION

Example embodiments will now be described more fully with reference to the accompanying drawings.

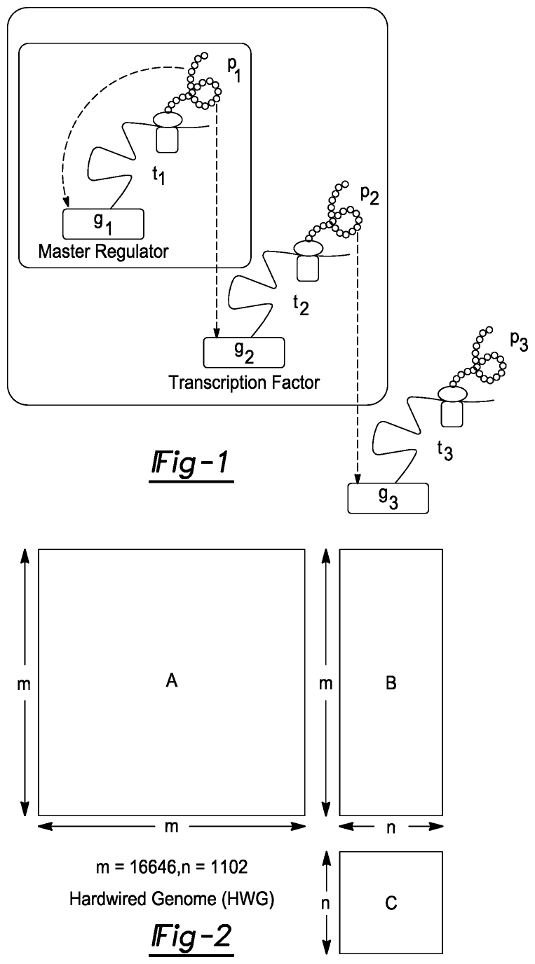

FIG. 1 illustrates a simplified network for the human genome. For simplicity, consider three genes in the human genome, G1, G2, and G3. G1 mRNA is T1, and the protein product of G1 is P1. G2 produces mRNA T2 and protein P2, and likewise for G3, T3, and P3. P1 localizes to the nucleus and regulates transcription of G1 and G2. P2 also localizes to the nucleus, but only regulates transcription of G3. Thus, one can define G1 and G2 as transcription factors; whereas G3 is not. G1 is special in that it is a master regulator transcription factor, which can be defined as a self-regulating transcription factor. If all genes are classified using this hierarchy, one can construct a universal gene network that is cell type invariant. Such a network is referred to herein as the Hardwired Genome (HWG).

FIG. 2 depicts an example embodiment for the Hardwired Genome which is a data-guided network construction of the human genome. The Hardwired Genome is comprised of three components: A-matrix; B-matrix; and C-matrix. The A-Matrix is a representation of all possible protein-protein interactions. The B-Matrix represents the transcription factor (TF)-DNA interactions. The C-Matrix represents the TF-TF interactions. Here, network and matrix are used interchangeably, as these have the same representation in mathematics.

More specifically, the Hardwired Genome is restricted to a curated set of protein-coding genes in the human genome. A-Matrix is an m×m matrix of protein-protein interactions. Edges are assigned a confidence score of 0-1000 (representing the probability of an interaction). In an example embodiment, if one thresholds at 600, m is 16646. This is the core data structure used in computing network features, such as eigenvector centrality (EC). B-Matrix is a 16646×1007 rectangular matrix of TF-DNA interactions. The data represents the known binding sites for transcription factors at gene transcription start site (TSS) (defined by the user) and are derived from both biological data and using bioinformatics. This is the core data structure used in cellular reprogramming predictions. C-Matrix is a 1007×1007 matrix of TF-TF regulatory interactions. The data represents binding sites and activity coefficients (if available) for transcription factors at different TSS locations for TF producing genes. This is the core data structure used for navigating the genome.

The Hardwired Genome is constructed using a combination of internal data and publicly available data sources. Although not limited hereto, example data sources are set forth in Table 1 below.

| Data Source | Description |
|---|---|
| STRING | Protein-protein interactions from 19,566 protein |
| | coding genes, derived from experimental data, |
| | computational perdictions, and mining of publicly |
| | available texts |
| FANTOMS | High resolution RNA-seq data (CAGE-seq) from |
| | 2,000 samples of over 200 cell types |
| GTEx | Tissue specific RNA-seq of 54 non-diseased |
| | tissue sites from almost 1000 individuals |
| HumanTF | 1,800 transcription factors and their binding |
| | motifs |
| Roadmap Epigenomics | Chromatin accessibility through DNase-seq |
| ENCODE | Chromatin accessibility through DNase-seq |
| The Human Reference | 64,006 experimentally validated protein-protein |
| Interactome (HuRI) | interations from 9,094 proteins |
| 4DNucleome Portal | Nucleomics data from almost 4,000 experiments |
| | covering over 1,500 experiment sets |
| KEGG | 537 curated biological, drug, and disease |
| | pathways |
| PANTHER | 177 curated biological, drug, and disease |
| | pathways |

Other data sources also fall within the scope of this disclosure.

One application for the Hardwired Genome is reprogramming cells. For this application, the objective is to mathematically identify a set of transcription factors that will directly reprogram a sample cell of a given cell type to a cell of a desired cell type. In an example embodiment, the problem is modeled with a discrete-time-invariant linear control system with the form

x(k+1)=Ax(k)+Bu(k) (1)

where x(k+1) is an output vector in the state-space representation, x(k) is a state vector in the state-space representation, A is the state transition matrix in the state-space representation, B is the input matrix in the state-space representation and u(k) is the input vector in the state-space representation. Further information regarding this control system approach, reference may be made to U.S. Pat. No. 10,672,501 which is incorporated in its entirety herein.

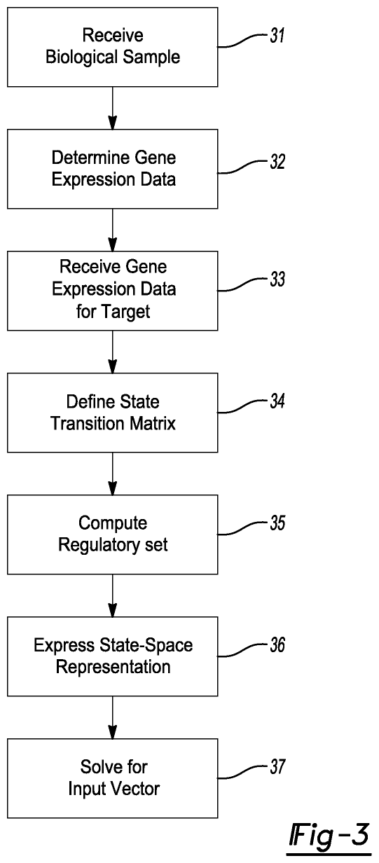

FIG. 3 provides an overview for a proposed method for reprogramming cells of a subject using this control system approach. A biological sample of a sample cell is first received at 31 from a subject, where the sample cell has a given cell type. The sample cell represents the initial state in the state-space model approach. In the example embodiment, the sample cell is a skin cell although other types of cells (e.g., embryonic cells) also fall within the scope of this disclosure.

From the biological sample, gene expression data is determined at 32 for the sample cell. In one embodiment, the gene expression data is further define as RNA-seq data which can be extracted from the biological sample using known DNA sequencing techniques. Other types of gene expression data include but are not limited to CAGE, Proteomics, Bru-seq, etc.

Likewise, gene expression data is received at 33 for a target cell having a target cell type, where the target cell type differs from the given initial cell type. In the example embodiment, target cell type is a muscle cell and the gene expression data for the target cell is also defined as RNA-seq data. Target cell types also include but are not limited to embryonic, cardiac, neuron, retinal, red blood cell, islets, T-cells, etc.

Next, the state transition matrix, A, which models cell dynamics is derived at 34. In one embodiment, a graph is constructed for the human genome, where each node in the graph represents a gene in the genome and each edge in the graph quantifies the relationship between two genes. Specifically, the graph is the Hardwired Genome described above. For use as the state transition matrix, an adjacency matrix is formed from the graph, i.e., the Hardwired Genome, and then used as the state transition matrix. In one embodiment, the A-matrix from FIG. 2 is used as the adjacency matrix.

In another embodiment, the state transition matrix may be tailored to a specific cell type. In place of the cell type invariant Hardwired Genome, a cell type specific Hardwired Genome is derived by evaluating the A-matrix, the B-matrix and the C-matrix using genomic data, such as RNAseq (gene expression) or DNAsel (accessibility of a gene for transcription) from a given cell type. From RNAseq and DNAsel, one can extract which genes (nodes) are inactive using a user define threshold. Inactive genes (nodes) and corresponding interactions (edges) are then masked to form a subgraph. In this way, a subgraph is extracted from the graph, i.e., the Hardwired Genome. This creates a new HWG that is cell type specific. Lastly, the adjacency matrix is formed from the subgraph (e.g., being equated to the A-matrix) and used as the state transition matrix.

Regulatory sets define where a given set of transcription factors could possibly influence the genome. The matrix B encodes where the control signal u[k] can influence the existing network defined by A. With b_{k,j} representing the regulation weight of TF j on TAD k, one can define an input matrix Bⱼ for each TF j:

Bⱼ:=(b_{k,j})∈ [symbol] ^{N×1}, k=1, . . . , N. (2)

In this way, the regulatory set is computed at 35 for one or more transcription factors. It is understood that a regulator set for a plurality of transcription factors can be formed by taking the union of the regulatory sets for each individual transcription factor.

Reprogramming of a sample cell to a target cell is expressed at 36 with a state-space representation of a linear system as given in equation (1) above, where the gene expression data for the target cell serves as an output vector in the state-space representation, the adjacency matrix serves as the state transition matrix, the gene expression data for the sample cell serves as a state vector in the state-space representation, the regulatory set for the given transcription factor serves as an input matrix in the state-space representation, and an input vector in the state-space representation represents the given transcription factor.

Lastly, the input vector in the state-space representation is solved for as indicated at 37. In a simplified embodiment, cells may be reprogrammed directly using one transcription factor. Because an input matrix B has been defined for each transcription factor, an input vector is determined for each transcription factor using the corresponding input matrix. The values for the input vector may be determined, for example using a least squares method executed in MATLAB. Other regression techniques may also be used to solve for the input vector in the state-space representation. The transcription factor which results in a cell that is closest to the target cell type is deemed the solution. Based on the solution, at least one transcription factor can be introduced and/or manipulated in a cell of the subject, where the cell has the given cell type and the at least one transcription factor is in the solution (i.e., input vector).

Imposing some biology into the algorithm, it is known that transcription factors used in direct reprogramming are almost always up-regulated in the target state. In order to choose transcription factors most reflective of reality and minimize computation time, subsets of transcription factors can be chosen for each direct reprogramming calculation before computation.

In an example embodiment, transcription factors are selected for the subset of transcription factors if they meet the following criteria. First, the transcription factor is expressed in the target cell. For example, greater than 4 RPKM expression in target state. This criterion helps to minimize potential noise in genomic signatures in TF subset selection. Second, the expression of the transcription factor in the target state must be greater than some threshold (e.g., 10) as compared to the initial state. This criterion is used to select transcription factors that are up-regulated in the target state. Rather than solving for all 300+ transcription factors, calculations can be made for only the transcription factors which meet the threshold criteria above. Other types of thresholding criteria are envisioned and fall within the broader aspects of this disclosure.

Different transcription factors can be input to the cell at different points through the cell cycle. In an example embodiment, there are five possible input times (i.e., 0, 8, 16, 24, and 32 hours) although more or less input times are possible. Once a transcription factor is input, it is assumed that it continues to influence the system until the end of the cell cycle (e.g., 40 hours).

The cellular reprogramming field faces a critical need to improve yield. The goal here is to demonstrate improved yield through refinement of choice and timing of transcription factors (TF), based on the results of a carefully designed sequence of experiments. The structure of the i^{th} experiment will be informed by existing databases and the results of earlier experiments in the sequence. The essence of the proposed approach is to find experimentally an approximation to the gradient of the yield function and adjust TF “recipes” while moving in the direction of the gradient.

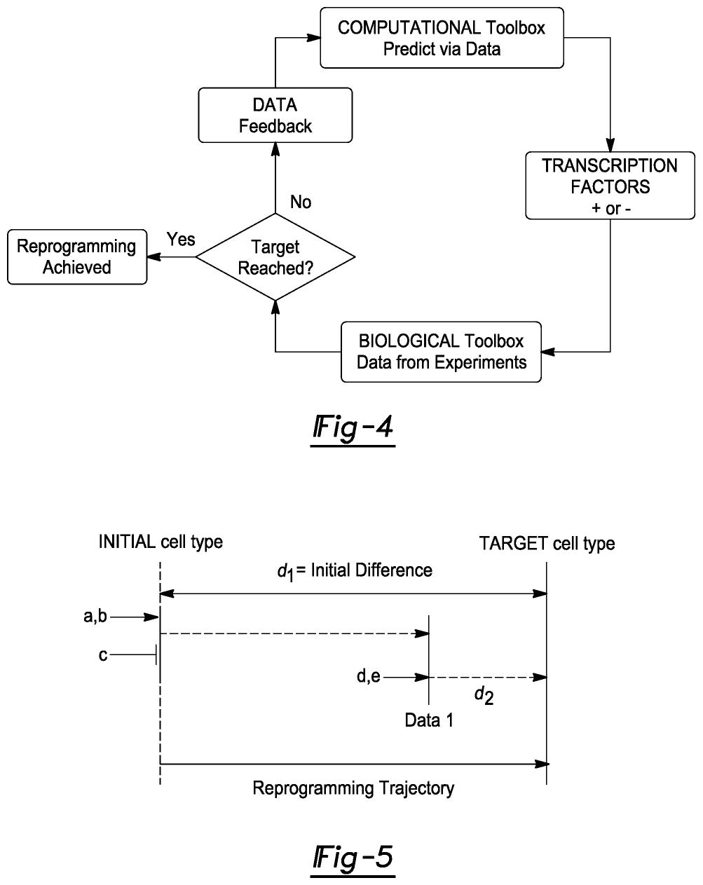

The flow of data to and from a computational toolbox 41 is shown in FIG. 4. Output from the computational toolbox, based on existing data, is a TF recipe for reprogramming one cell type into another. Predicted transcription factors are then tested experimentally. Phenotype data collected from cells treated with the transcription factors are fed back into the computational toolbox if reprogramming is incomplete.

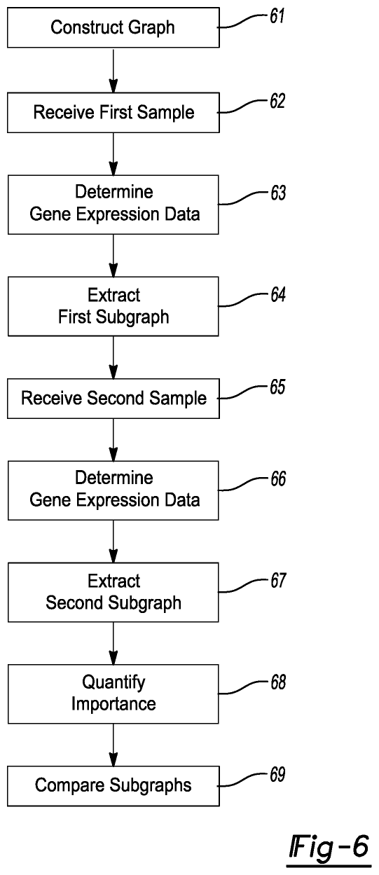

FIG. 5 shows the initial cell type, the target cell type, and intermediate cell types along the cell reprogramming trajectory. d₁ is the difference between initial and target cell types, which is used to generate a transcription prediction. Treatment with predicted transcription factors a and b, and suppression of transcription factor c, reprograms the cells to an intermediate cell type indicated at Data 1. Data from this intermediate are used to find d₂, the distance between the intermediate and target cell types. Based on d₂, the transcription factors d and e are predicted to improve reprogramming to the target state.

Cell programming is one application for the proposed network approach to analyzing the human genome. The proposed cell type invariant Hardwired Genome and the cell type specific Hardwired Genome can be extended to other applications as well.

FIG. 6 presents a method for analyzing a genome of a cell. As a starting point, a cell type invariant graph representing the genome is constructed at 61, where each node in the graph represents a gene in the genome and each edge in the graph quantifies the relationship between two genes. The graph may be constructed in the manner described above and is referred to herein as the Hardwired Genome.

To analyze a cell of a given cell type, a biological sample for a first cell of a first cell type is obtained at 62. From the biological sample, gene expression data for the first cell is then determined at 63. In one embodiment, the gene expression data is further define as RNA-seq data which can be extracted from the biological sample using known DNA sequencing techniques. Other types of gene expression data include but are not limited to CAGE, Proteomics, Bru-seq, etc.

Next, a first subgraph is extracted from the graph at 64 using the gene expression data for the first cell, where the first subgraph represents the first cell type. In one embodiment, inactive genes (i.e., nodes in the graph) are identified using thresholding and the identified genes along with corresponding interactions (i.e., edges in the graph) are removed from the graph to form the first subgraph.

To compare the first cell to another cell having a different cell type, similar steps may be applied to a biological sample for a second cell. That is, gene expression data is determined at 66 for the second cell, and a second subgraph is extracted from the graph at 67 using the gene expression data. The result is a first subgraph indicative of the first cell type and a second subgraph indicative of the second cell type. In some embodiments, these two subgraphs can be compared to each other, for example by computing a distance between the two subgraphs.

Before comparing the subgraphs, the importance of the nodes in the subgraphs may be quantified at 68 using centrality. Unlike other methods, centrality identifies a subset of nodes in a graph having the greatest importance. In one example, a page rank algorithm can be applied to each of the subgraphs, thereby yielding an eigenvector which represents and quantifies the importance of each node in the subgraphs. The two subgraphs are then compared at 69 by computing a distance between the eigenvectors. Other techniques for comparing the subgraphs are contemplated by this disclosure. Likewise, other types of centrality concepts, including but not limited to degree centrality, closeness centrality, and betweenness centrality, can be applied to the two subgraphs.

Comparing two subgraphs can be very beneficial for understanding the human genome and used in different applications. One suitable application is cell reprogramming, where the comparison may be helpful, for example in selecting a subgraph as the adjacency matrix as described above.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for reprogramming cells of a subject, comprising:
receiving a biological sample of a sample cell from the subject, where the sample cell has a given cell type;
determining gene expression data for the sample cell from the biological sample;
constructing a graph for a genome, where each node in the graph represents a gene in the genome and each edge in the graph quantifies the relationship between two genes;
forming an adjacency matrix from the graph;
receiving gene expression data for a target cell having a target cell type, where the target cell type differs from the given cell type;
computing a regulatory set for a set of transcription factors, where the regulatory set quantifies influence of the transcription factors in the set of transcription factors on a genome;
expressing reprogramming of the sample cell to the target cell with a state-space representation of a linear system, where the gene expression data for the target cell serves as an output vector in the state-space representation, the adjacency matrix serves as a state transition matrix, the gene expression data for the sample cell serves as a state vector in the state-space representation, the regulatory set for the given transcription factor serves as an input matrix in the state-space representation, and an input vector in the state-space representation represents the given transcription factor;
solving for the input vector in the state-space representation; and
manipulating at least one transcription factor in a particular cell of the subject, where the particular cell has the given cell type and the at least one transcription factor is in the input vector.

2. The method of claim 1 further comprises constructing a graph for a genome by representing protein-protein interactions, transcription-DNA interactions and transcription factor-transcription factor interactions with a series of matrices.

3. The method of claim 1 further comprises identifying a subset of vertices in the graph based on centrality.

4. The method of claim 3 further comprises identifying a subset of vertices in the graph using one of degree centrality, closeness centrality, betweenness centrality and eigenvector centrality.

5. The method of claim 1 further comprises extracting a subgraph from the graph and forming the adjacency matrix from the subgraph, where the subgraph represents a specific cell type.

6. The method of claim 1 wherein the gene expression data for the sample cell is further defined as RNA-seq data.

7. The method of claim 1 wherein computing a regulatory set for a set of transcription factors further comprises computing a regulator set for each of a plurality of transcription factors and joining the plurality of regulatory sets to form the input matrix.

8. The method claim 1 wherein solving for the input vector further comprises determining values for the input vector that minimize distance between the sample cell and the target cell.

9. The method of claim 8 further comprises determining values for the input vector using a least squares method.

10. The method of claim 1 wherein manipulating at least one transcription factor includes at least one of introducing a given transcription factor into the particular cell or removing the given transcription factor from the particular cell.

11. A computer-implemented method for modeling the genome of a cell, comprising:
constructing a graph for a genome, where each node in the graph represents a gene in the genome and each edge in the graph quantifies the relationship between two genes;
receiving a first biological sample of a first cell of a subject, where the first cell has a first cell type;
determining gene expression data for the first cell from the first biological sample;
extracting a first subgraph from the graph using the gene expression data for the first cell, where the subgraph represents the first cell type;
receiving a second biological sample of a second cell of a subject, where the second cell has a second cell type;
determining gene expression data for the second cell from the second biological sample;
extracting a second subgraph from the graph using the gene expression data for the second cell, where the second subgraph represents a second cell type; and
comparing the first subgraph to the second subgraph.

12. The method of claim 11 further comprises quantifying importance of nodes in the first and second subgraphs using centrality before the step of comparing the first subgraph to the second subgraph.

13. The method of claim 12 further comprises quantifying importance of nodes in the first and second subgraphs by applying a page rank method to the first and second subgraphs and computing a distance between eigenvectors associated with the first and second subgraphs.

14. The method of claim 11 further comprises constructing a graph for a genome by representing protein-protein interactions, transcription-DNA interactions and transcription factor-transcription factor interactions with a series of matrices.

15. The method of claim 12 wherein the gene expression data for at least one of the first cell or the second cell is further defined as RNA-seq data.
